(19) United States
(12) Patent Application Publication
(10) Pub. No.: US 2024/0058131 A1
(43) Pub. Date: Feb. 22, 2024

(54) A METHOD OF MAKING AN INDIVIDUAL 3D PRINTED CERAMIC BIORESORBABLE BONE IMPLANT FOR USE IN TRAUMATOLOGY AND ORTHOPEDICS

(71) Applicant: Advanced Development of Additive Manufacturing, Inc., Stamford, CT (US)

(72) Inventors: Arkadii Bohdan, Lymanka Village (UA); Vadym Volkov, Kryzhanivka Village (UA); Oleg Rogankov, Odessa (UA)

(21) Appl. No.: 18/041,333

(22) PCT Filed: Aug. 10, 2021
(86) PCT No.: PCT/US21/45382

§ 371 Date: Feb. 10, 2023

Related U.S. Application Data
(60) Provisional application No. 63/063,483, filed on Aug. 10, 2020.
(60) Provisional application No. 63/063,485, filed on Aug. 10, 2020.

(51) Int. Cl. A61F 2/30; A61L 27/58; A61L 27/10; A61L 27/42; A61L 27/20; B33Y 80/00; B33Y 10/00; B33Y 70/10

(52) U.S. Cl. CPC A61F 2/30942; A61L 27/58; A61L 27/10; A61L 27/425; A61L 27/20; B33Y 80/00; B33Y 10/00; B33Y 70/10; A61F 2002/30943; A61F 2002/30985; A61F 2002/30968

(57) ABSTRACT

The present invention relates to ceramic bioresorbable bone implants made from a material based on a glass-ceramic and/or polysaccharide and/or a calcium-based mineral. The proposed composition is suitable for 3D printing. The bone implants are used in traumatology and orthopedics for treatment of bone diseases. The proposed composition of the implant provides osteoinductive and osteoconductive activity at the site of transplantation, with the subsequent replacement of the implant with native bone tissue.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

Tissue engineering is an interdisciplinary field that applies the principles of engineering, medicine, basic sciences to develop tissue substitutes—implants that restore, maintain or improve the function of human tissues. Large-scale cultivation of human, animal or artificial origin cells can provide materials to replace damaged components in humans.

Natural or synthetic materials when implanted into the human body as temporary structures provide a framework that allows the body's own cells to migrate, differentiate, grow and form new tissues, while the framework itself is gradually absorbed by the body. The requirements for ceramic implants are: high porosity, an open pore network for cell growth and transfer of nutrients and metabolic waste; biocompatibility and bioresorbability with controlled rates of decomposition and resorption to match the rate of tissue replacement in the body; suitable surface composition for cell attachment, proliferation and differentiation; mechanical properties corresponding to the tissues at the site of implantation.

The structure of the implant must protect the interior of the proliferating cells of the pore network and their extracellular matrix from mechanical overload for a sufficient period of time. This is especially important for supporting tissues such as bones and cartilage.

### BACKGROUND

Currently, the treatment of patients who have suffered injuries that include complex bone fractures that cannot be repaired includes the use of non-degradable fixation elements (such as titanium plates, pins, etc.) that generally may require additional invasive procedures reoperation to remove or adjust them.

The biodegradable properties of the implant in the present invention allow the patient to recover with one operation without the need for additional medical measures as opposed to using metal-containing (e.g. Ti) implants that generally might lead to long-term issues, especially in cases where patients are immunosuppressant.

### BRIEF DESCRIPTION OF THE DRAWINGS

The present embodiments are illustrated by way of example and are not intended to be limited by the figures of the accompanying drawings.

FIG. 1 is an example of a 3d-printed implant of a patella according to the present invention.

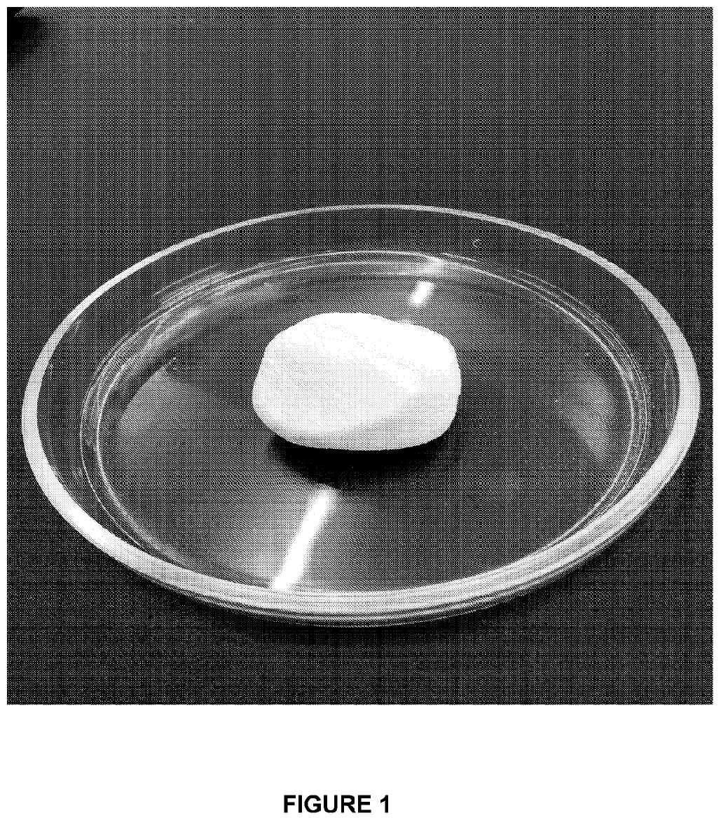

FIG. 2 is an example design of the implant of a patella according to the present invention.

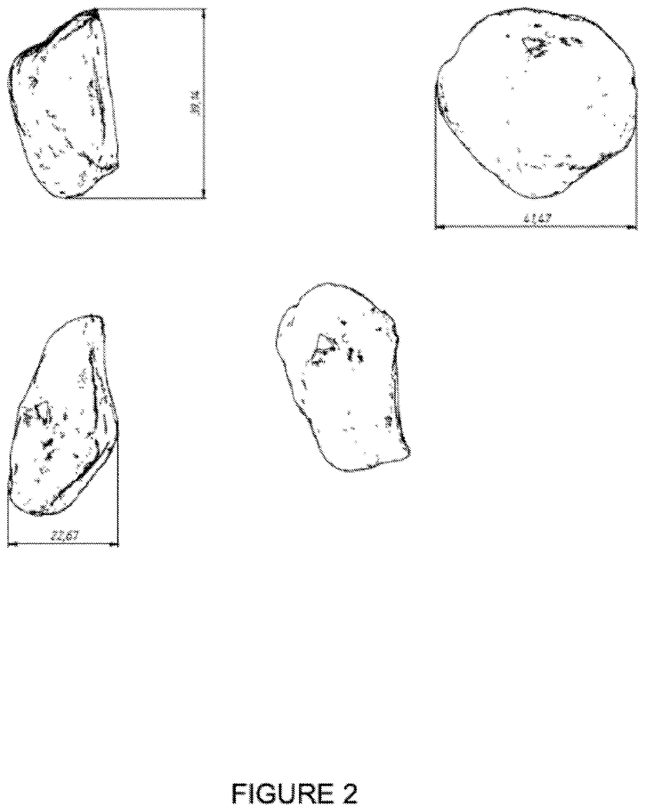

FIG. 3 is an example design of a proximal part of the radial bone with the head of the radial bone according to the present invention.

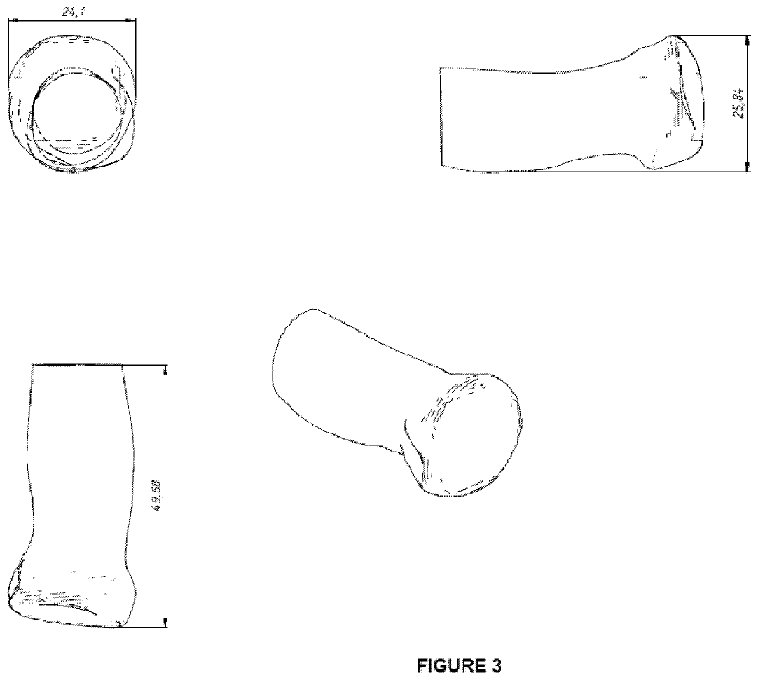

FIG. 4 is an example of a printed proximal part of the radial bone with the head of the radial bone according to the present invention.

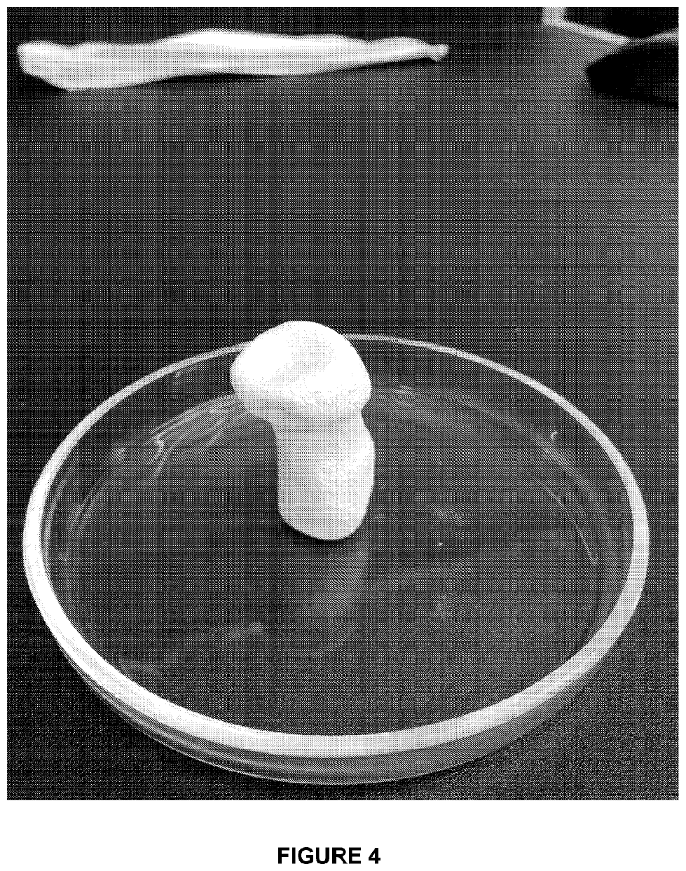

FIG. 5 is an example process for replacing a bone defect using the method and composition of the present invention.

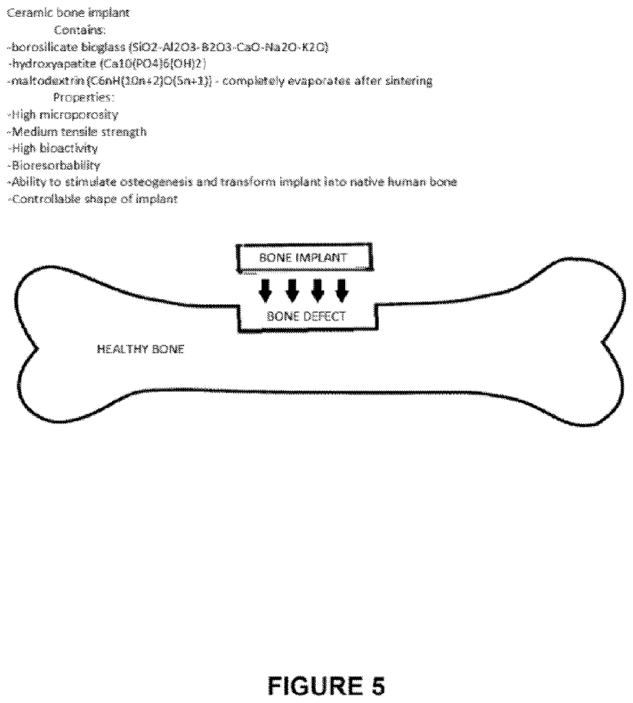

### BRIEF DESCRIPTION OF THE INVENTION

The implants of the present invention are adopted by and transformed by human tissue into human bone and do not require repeated surgical intervention, thereby improving the quality of treatment and facilitating the patient's healing process.

The present invention eliminates the need to traumatize a patient for the purpose of bone marrow autoplasty.

The method and resulting mold of the present invention provides sufficient strength indicators to withstand implant loads for use in orthopedic surgery.

The composition of the present invention was methodically selected so that it doesn't have allergic, toxic, oncogenic properties.

The 3D modeling technique of the present invention allows the medical community to perform an implant in any size and shape, taking into account the need for a particular clinical case.

Biodegradable materials, irrespective of their constituent form, are expected to degrade progressively over a period of time to assist as scaffolds or for the healing process.

### Definitions

The term “implant” is intended to describe one of the well-known shaped transplants to be placed into the bone defect.

The term “biocompatible” is intended to describe the ability of material to perform as a substrate that will support the appropriate cellular activity, including the facilitation of molecular and mechanical signalling systems, in order to optimise tissue regeneration, without eliciting any undesirable effects in those cells, or inducing any undesirable local or systemic responses in the eventual host.

By the term “borosilicate glass” is intended powder mix of Silica oxide(IV), Aluminum oxide(III), Barium oxide(III), Calcium oxide(II), Sodium oxide, Potassium oxide in designated proportions.

By the term “bioresorbable” is intended the property of the material to be fully resorbed by the body enzymes to the simple metabolites that are presented in the human body.

Ceramic properties. By the term “ceramic properties” is intended the state of the product that is near to an inorganic compound of metal, metalloid and nonmetal with ionic or covalent bonds.

### DETAILED DESCRIPTION

The present invention method utilizes a binder-jet printing method. The method is one of a number of additive manufacturing processes that can be used to form three-dimensional objects such as medical implant molds by controlled injection of a binder from a moving print head onto layers of ceramic powder fed by a system of moving platforms and a print shaft. The print head produces movements along the X and Y axes.

The movement along the Z axis occurs due to the synchronous movement of the moving platforms. The feed platform rises to a fixed height up, the shaft takes the required amount of powder and evenly applies it to the printing surface, then the print head injects binder at specified points with high accuracy, the printing platform plunges down a fixed distance and the process repeats. The printer draws the engineered model one layer at a time.

The BJP method involves extruding the binder through a nozzle and binding the powder base at the injection site. The nozzle is part of the extruder head, the binder is fed into the print head through the feed system from the reservoir.

### Method Embodiment I

In one embodiment, the present method of making a ceramic powder for 3D printing includes mixing an initial powder base mixture comprising the composition having 64% of hydroxyapatite with 27% of borosilicate glass and with 9% of maltodextrin by loading into a V-shaped mixer and mixing it for 2 hours. The obtained mixed printing powder is loaded into the 3D-printer feed chamber to make the 3D printed mold.

In the powder composition represented in Table 1, the calcium hydroxyapatite is the chemical compound of chemical formula Ca₁₀ (PO₄)₆ (OH)₂ wherein the Ca/P ratio is 1.63-1.75.

The borosilicate glass is the chemical compound of SiO₂ Al₂O₃B₂O₃ CaO Na₂O K₂O. And, maltodextrin is a polysaccharide that is produced from vegetable starch by partial hydrolysis, wherein maltodextrin has the chemical formula of C₍₆ₙ₎ H₍₁₀ₙ₊₂₎ O₍₅ₙ₊₁₎.

The present invention uses a binder solution used for extruding onto a printing surface via the printing head to bind powder particles in designated locations. The binder composition is 40 vol % of ethylene glycol, 10 vol % of isopropyl alcohol, 10 vol % of glycerin, 3 vol % of cocoate, and 300 vol % of distilled water plus remainder proportion of impurities.

To make an implant, the design begins with creating a 3D model. A patient's CT/MRI images of the damaged area are processed using specialized program software; localization and size of the injury are assessed; and, on the basis of the obtained data, a 3D image of the symmetrical bone is taken from the other side and mirrored. If the fracture of the right humerus requires a repair, then an observation is made of the necessary area of the left humerus with the mirror image.

Alternatively, a CT/MRI scan of the patient is taken before an injury; the resulting image is cropped to include only the necessary anatomical structures. Then the obtained 3D model is converted to an “.stl” format and is prepared for further printing. During all the manipulations, the scale and size of the 3D model are preserved. The model is imported into the software of a 3D printer to further the printing. A standard software package automatically generates support when needed. An example of the support might be in the additional construction model, which fixates the model during the printing and sintering processes and does not connect with the body of an implant.

After printing, the resulting object remains in the printing chamber for 8 hours for better binding, and for better interconnectedness between powder particles after the printing process. Then the object is dried in a drying chamber at 80° C. for 8 hours to evaporate excess binder from the object. Next comes the process of dedusting.

Dedusting is the removal of excess powder from the surface of the object using a spray gun with compressed air and a set of brushes. Then the object is sent for firing in a muffle furnace at a peak temperature of 1150° C. for 12 hours. After firing, the finished implant is in the form of a monolithic structure with an open porous system. Information for optimal processing parameters is described in Table 2.

Open porosity is achieved due to the presence of maltodextrin in the composition. It is used to bind the powder base by injecting a binder with which the maltodextrin comes into contact and holds the surrounding powder particles together. Since the decomposition temperature of maltodextrin is 350° C., during firing, it decomposes and leaves cavities that are interconnected and form an open pore system.

The three-dimensional ceramic matrix of the implant has the kinetics of degradation and resorption up to 18 months and the ability to maintain a given shape under the action of biomechanical stress. Such a composite material improves the biocompatibility and integration of hard tissues. Hydroxyapatite provides an artificial bone matrix which stimulates the vascularisation and differentiation of macrophages and mesenchymal stem cells to osteogenic cells and is neutral for the human body.

In addition, the main products of hydroxyapatite resorption help to buffer the by-products of the acidic resorption of aliphatic polyester and thereby help avoid the formation of an unfavorable environment for the cells of hard tissues due to the low pH. The components of borosilicate glass provide increased bioactivity of the implant and stimulate the growth of osteoblasts on its surface. Final result of the treatment with the proposed implant is completely regenerated bone in the place of former bone defect.

### Composition Embodiment I

The present invention uses binder-jet printing to process a bioresorbable composite of two biomaterials, borosilicate glass and hydroxyapatite, to meet all the criteria for use in tissue engineering applications.

TABLE 1
Exemplary embodiment with percentages
of the composition and its benefits:

| | | Preferred | Benefit of optimal |
|---|---|---|---|
| Component | Range | percentage | percentage |
| hydroxyapatite | 60-70% | 64% | Optimal cell activity |
| borosilicate | 25-35% | 27% | Density is >70 MPa |
| glass/45S5 Bioglass | | | |
| maltodextrin | 5-15% | 9% | Optimal amount for |
| | | | printing and provides |
| | | | open-pore structure |
| | | | after sintering |

Hydroxyapatite range of <60% yields a mold that has inefficient properties, because the implant shows low cell activity according to the tests.

Hydroxyapatite range of >70% yields a mold that has inefficient properties, because the implant has too low a density according to the tests.

Borosilicate glass/45S5 Bioglass range of <25% yields a mold that has inefficient properties, because the implant does not properly sinter and loses its structure according to the tests.

Borosilicate glass/45S5 Bioglass range of >35% yields a mold that has inefficient properties, because the implant shows low cell activity according to the tests.

Maltodextrin range of >5% yields a mold that has inefficient properties, because the printing powder at the printing stage is not held together by a binder according to the tests.

Maltodextrin range of <10% yields a mold that has inefficient properties, because the implant loses its structure according to the tests.

### Method Embodiment II

TABLE 2
Exemplary embodiment with processing parameters:

| | | Preferred | |
|---|---|---|---|
| Process | Range | Value | Benefits & goals |
| Mixing (min) | 30-360 | 120 | printing powder |
| | | | homogenization |
| | | | (which is critical for |
| | | | printing quality) |
| Drying time (min) | 30-720 | 480 | vaporizing of binder |
| | | | from implant |
| | | | structure |
| Drying temperature | 20-150 | 80 | vaporizing of binder |
| (degrees C.) | | | from implant |
| | | | structure |
| Sintering | 600-1200 | 1150 | required condition |
| temperature peak | | | for chemical |
| (degrees C.) | | | reactions in the |
| | | | composition |
| Sintering time(min) | 120-1080 | 720 | necessary condition |
| | | | for chemical |
| | | | reactions in the |
| | | | composition |
| Sintering regime | 1-20° C. | 5 | necessary condition |
| | per | | for chemical |
| | minute | | reactions in the |
| | temperature | | composition |
| | rise | | |

Mixing time of <30 min yields a mold that has inefficient properties, because printing powder remains non-mixed which leads to poor printing quality according to tests.

Mixing time of >360 min yields a mold that has inefficient properties, because after this point (at 360 min) the maximum homogenization of powder has been achieved and further mixing degrades the porosity and density of the mold according to the tests.

Sintering temperature of <600° C. yields a mold that has inefficient properties, because such a temperature limits the necessary chemical reactions, which enable the ceramic properties according to the present invention.

Sintering temperature of >1200° C. yields a mold that has inefficient properties, because hydroxyapatite crystallizes after 1200° C. which leads to a loss of biologically active properties of hydroxyapatite according to the tests.

Sintering time of <120 min yields a mold that has inefficient properties, because the furnace cannot reach the peak temperature required for sintering according to the tests.

Sintering time of >720 min yields a mold that has inefficient properties, because it leads to degradation of chemical composition and deformation of the implant according to the tests.

### Exemplary Method

The system of the present invention is described as follows:
V-shaped mixer
Binder-jet Ceramic 3D-printer—ZCorp Ceramic 3D Printer Zprinter 310/Kwambio Ceramo Zero Max™.
Drying cabinet
Muffle Furnace

In the V-shaped mixer, adding the powder base comprising the composition having 64% hydroxyapatite with 27% of borosilicate glass and 9% maltodextrin, and mixing for 2 hours.

In addition, preparing a binder in composition comprising 40 vol % of ethylene glycol, 10 vol % of isopropyl alcohol, 10 vol % of glycerin, 3 vol % of cocoate, 300 vol % of distilled water in designated proportions.

The 3D implant printing steps:
a. Setup of the 3D printer with the preset parameters
b. Simulation of a 3D model in Autodesk 3DS Max using the results of a patient's CT/MRI examination
c. Loading the printing powder into the printer feed chamber.
d. Downloading 3D model to a printer
e. Printing the implant
f. Holding the implant in the mass of the printed powder for better binding of particles for 8 hours
g. Drying the implant in the drying chamber at 80° C. for 2 hours
h. Removing the excess powder from the surface of the implant using a spray gun with compressed air and a set of brushes
i. Sintering the implant in a muffle furnace at 1150° C. with an increase to a peak temperature for 12 hours
j. Cooling the furnace for 12 hours, removing the implant from furnace
k. Sterilizing the implant and packaging
l. Implanting surgically to the site of a bone defect according to traumatologic methods of surgical interventions

TABLE 3
Exemplary properties of bioresorbable implant:

| | 64% of hydroxyapatite; 27% of |
|---|---|
| | borosilicate glass; 9% of |
| Composition: | maltodextrin |
| Density | up to 3.15 g/cm³ |
| Total porous volume | up to 15% |
| Pore diameter | up to 350 nm |
| Pore | connected network |
| interconnectedness | |
| Compressive | up to 60 mPa |
| strength | |
| Tensile strength | up to 85 mPa |
| Bending strength | up to 50 mPa |
| pH testing | up to 6.3 |
| Dissolution/solubility | dissolution after |
| testing | approximately 34 weeks |

Any of the steps as described in any methods or flow processes herein can be performed in any order to the extent the steps in the methods or flow processes remain logical.

Note that any and all of the embodiments described above can be combined with each other, except to the extent that it may be stated otherwise above or to the extent that any such embodiments might be mutually exclusive in function and/or structure.

Although the present invention has been described with reference to specific exemplary embodiments, it will be recognized that the invention is not limited to the embodiments described but can be practiced with modification and alteration within the spirit and scope of the appended claims. Accordingly, the specification and drawings are to be regarded in an illustrative sense rather than a restrictive sense.

What is claimed is:

1. A method for producing a bioresorbable implant comprising:
preparing a powder base comprising up to 70% hydroxyapatite, up to 40% borosilicate glass and up to 20% maltodextrin;
homogenizing the powder base by loading the powder base into a V-shaped mixer and mixing for up to 360 min;
preparing a binder comprising having up to 50 vol % of ethylene glycol, up to 20 vol % isopropyl alcohol, up to 20 vol % glycerin, up to 10 vol % cocoate, and up to 400 vol % distilled water;
loading the powder base and the binder into a ceramic 3D-printer, wherein the 3D-printer output material includes at least one of a glass-ceramic, a polysaccharide, and/or a calcium-based mineral;
creating a 3D-model of an implant for printing;
printing the 3D-model of the implant using the ceramic 3D-printer to make a printed implant;
holding the printed implant in a printing chamber for up to 720 min;
drying the printed implant in a drying chamber at up to 150° for up to 720 min to make a dried implant;
removing excess powder from the surface of the dried implant using a spray gun with compressed air and a set of brushes;
sintering the dried implant in a muffle furnace at up to 1200° C. peak temperature for up to 1080 min;
cooling the sintered implant in the muffle furnace for up to 1080 min.

2. The method according to claim 1, wherein a calcium-based mineral used in the ceramic 3D printer is selected from the group of chemical compounds consisting of calcium and/or phosphorus with the Ca/P ratio of 1.5-1.67, tricalcium phosphate, monocalcium phosphate, dicalcium phosphate, tetracalcium phosphate, hydroxyapatite, alpha-tricalcium phosphate, beta-tricalcium phosphate, calcium oxide(II) and mixtures thereof.

3. The method according to claim 1, wherein the glass-ceramic is selected from a group of chemical compounds consisting of: silica oxide, calcium oxide, calcium chloride, calcium phosphate, calcium hydrophosphate, phosphorus oxide, barium oxide, barium chloride, barium phosphate, barium hydrophosphate, alumina oxide, alumina chloride, potassium oxide, potassium chloride, potassium hydrocarbonate, sodium oxide, sodium chloride, sodium hydrocarbonate and mixtures thereof.

4. The method according to claim 1, wherein the polysaccharide is selected from a group of chemical compounds comprising starch-based maltodextrin, dextrin, dextran, isomaltooligosaccharide, pectin, chitosan and mixtures thereof.

5. The method according to claim 1, wherein the powder base comprises 64% hydroxyapatite, 27% borosilicate glass and 9% maltodextrin.

6. The method according to claim 1, wherein the powder base is mixed in the V-shaped mixer for 120 minutes.

7. The method according to claim 1, wherein the binder comprises 40 vol % ethylene glycol, 10 vol % isopropyl alcohol, 10 vol % glycerin, 3 vol % cocoate, 300 vol % distilled water.

8. The method according to claim 1, wherein the printed implant is held in the printing chamber for 480 min.

9. The method according to claim 1, wherein the printed implant is dried in the drying chamber at 80°.

10. The method according to claim 1, wherein the printed implant is dried in the drying chamber for 480 min.

11. The method according to claim 1, wherein the dried implant is sintered in the muffle furnace at a peak temperature of 1150° C.

12. The method according to claim 1, wherein the printed implant is sintered in the muffle furnace for 720 min.

13. The method according to claim 1, wherein the muffle furnace is cools for 720 min.

14. The method according to claim 1, wherein the implant comprises 64% hydroxyapatite, 27% borosilicate glass, and 9% maltodextrin.

15. The method according to claim 14, wherein the peak temperature of the muffle furnace is 650° C. for 720 minutes.

16. The method according to claim 1, wherein the composition of the powder base comprises up to 95% 45S5 bioglass and up to 20% maltodextrin.

17. The method according to claim 1, wherein the composition of the powder base comprises up to 95% hydroxyapatite and up to 20% maltodextrin.

18. The method according to claim 1, wherein the composition of the powder base comprises up to 70% hydroxyapatite, up to 40% 45S5 bioglass and up to 20% maltodextrin.

19. A method for producing an implant comprising:
mixing a powder base comprising up to 30% hydroxyapatite and up to 70% borosilicate glass to make a mixture;
mixing the mixture in a V-shaped mixer for up to 120 min;
loading the powder base and a binder into a selective laser sintering 3D-printer;
receiving a collection of CT/MRI scans;
creating a 3D-model of an implant based on the collection of CT/MRI scans;
printing the 3D-model of the implant in the selective laser sintering 3D-printer;
removing excess powder from a surface of the printed implant by dedusting the printed implant using a spray gun compressed air and a set of brushes.

20. The method according to claim 19, wherein the powder base comprises up to 40% hydroxyapatite and up to 100% borosilicate glass.
